(19) United States
(12) Patent Application Publication
(10) Pub. No.: US 2022/0414298 A1
(43) Pub. Date: Dec. 29, 2022

(54) SIMULATION METHOD, SIMULATION APPARATUS, AND NON-TRANSITORY COMPUTER READABLE MEDIUM STORING PROGRAM

(71) Applicant: SUMITOMO HEAVY INDUSTRIES, LTD., Tokyo (JP)

(72) Inventor: Yoshitaka Kobayashi, Kanagawa (JP)

(21) Appl. No.: 17/825,736

(22) Filed: May 26, 2022

(30) Foreign Application Priority Data
Jun. 29, 2021 (JP) 2021-107625

(51) Int. Cl. G06F 30/25; G06F 17/11

(52) U.S. Cl. CPC G06F 30/25; G06F 17/11; G06F 2119/14

(57) ABSTRACT

Provided is a simulation method that represents an elastic analysis object as an aggregate of a plurality of virtual particles, and the method includes, when solving the equation of motion for each particle, regarding the analysis object as a rigid body, and calculating a force acting on the analysis object and a velocity of the analysis object, based on the force acting on each particle obtained at each time step and the velocity and the position of each particle, applying FIRE to a motion of the analysis object to calculate a force to be applied to the analysis object, obtaining a force to be additionally applied to each particle, by distributing the force to be applied to the analysis object to the plurality of particles, and solving the equation of motion, by adding the force to be additionally applied, to the force acting on each particle.

### RELATED APPLICATIONS

The content of Japanese Patent Application No. 2021-107625, on the basis of which priority benefits are claimed in an accompanying application data sheet, is in its entirety incorporated herein by reference.

### BACKGROUND

### Technical Field

A certain embodiment of the present invention relates to a simulation method, a simulation apparatus, and a non-transitory computer readable medium storing a program.

### Description of Related Art

Molecular dynamics method (MD method) and Renormalized Molecular Dynamics method (RMD method) are known as methods for simulating the motion of a plurality of particles in the related art. In the present specification, the molecular dynamics method and the renormalized molecular dynamics method are simply referred to as “molecular dynamics”. By generating an analysis model that represents an elastic analysis object with a plurality of particles, defining the forces acting between the particles, and analyzing the motions of these particles using the molecular dynamics, it is possible to simulate the behavior of analysis objects.

Fast Inertial Relaxation Engine (FIRE) is known as a method for obtaining a solution at high speed by converging the oscillation of the physical quantity to be obtained in a short time when performing static analysis of a particle system using a molecular dynamics (Erik Bitzek rt. al., “Structural Relaxation Made Simple”, Physical Review Letters 97, 170201 (2006)).

### SUMMARY

According to an embodiment of the present invention, there is provided a simulation method that represents an elastic analysis object as an aggregate of a plurality of virtual particles,

obtains a force acting on each particle at each time step, and

updates a velocity and a position of each particle by solving an equation of motion for each particle, based on the force acting on each particle, the simulation method including:

when solving the equation of motion for each particle,

regarding the analysis object as a rigid body, and calculating a force acting on the analysis object and a velocity of the analysis object, based on the force acting on each particle obtained at each time step and the velocity and the position of each particle;

applying FIRE to a motion of the analysis object to calculate a force to be applied to the analysis object;

obtaining a force to be additionally applied to each particle, by distributing the force to be applied to the analysis object to the plurality of particles; and

solving the equation of motion, by adding the force to be additionally applied, to the force acting on each particle.

According to another embodiment of the present invention,

there is provided a simulation apparatus that simulates a behavior of an elastic analysis object using a molecular dynamics method, the simulation apparatus including:

an input unit to which a simulation condition is input; and

a processing unit that performs analysis, based on the simulation condition input to the input unit, in which

the processing unit

represents the analysis object as an aggregate of a plurality of virtual particles,

obtains a force acting on each particle at each time step,

updates a velocity and a position of each particle by solving an equation of motion for each particle, based on the force acting on each particle, and

when solving the equation of motion for each particle,

regards the analysis object as a rigid body, and calculates a force acting on the analysis object and a velocity of the analysis object, based on the force acting on each particle obtained at each time step and the velocity and the position of each particle,

applies FIRE to a motion of the analysis object to calculate a force to be applied to the analysis object,

obtains a force to be additionally applied to each particle, by distributing the force to be applied to the analysis object to the plurality of particles, and

solves the equation of motion, by adding the force to be additionally applied, to the force acting on each particle.

According to still another embodiment of the present invention, there is provided a non-transitory computer readable medium storing a program causing a computer to execute a process including:

acquiring a simulation condition;

representing an elastic analysis object as an aggregate of a plurality of virtual particles;

obtaining a force acting on each particle at each time step;

updating a velocity and a position of each particle by solving an equation of motion for each particle, based on the force acting on each particle; and

when solving the equation of motion for each particle,

regarding the analysis object as a rigid body, and calculating a force acting on the analysis object and a velocity of the analysis object, based on the force acting on each particle obtained at each time step and the velocity and the position of each particle;

applying FIRE to a motion of the analysis object to calculate a force to be applied to the analysis object;

obtaining a force to be additionally applied to each particle, by distributing the force to be applied to the analysis object to the plurality of particles; and

solving the equation of motion, by adding the force to be additionally applied, to the force acting on each particle.

### BRIEF DESCRIPTION OF THE DRAWINGS

FIG. 1A is a schematic diagram showing an analysis model in which an elastic analysis object is represented by an aggregate of a plurality of particles, and FIG. 1B is a schematic diagram showing a physical quantity defined for the analysis object regarded as a rigid body.

FIG. 2 is a block diagram of a simulation apparatus according to a present embodiment.

FIG. 3 is a flowchart of a simulation method according to the present embodiment.

FIG. 4 is a graph showing a temporal change of a torque applied to a fixing member that fixes a part of a region of one end surface of a rotating body.

FIG. 5 is a graph showing a temporal change of an angular velocity co when the rotating body is regarded as a rigid body.

FIGS. 6A and 6B are schematic diagrams of an analysis model to be analyzed by applying FIRE.

### DETAILED DESCRIPTION

FIRE is applied to a single particle and cannot be applied to an aggregate of particles representing an elastic analysis object. It is desirable to provide a simulation method, a simulation apparatus, and a program that represent an elastic analysis object as an aggregate of a plurality of particles, and capable of obtaining a solution at high speed when solving the equation of motion for each particle to simulate the behavior of the analysis object.

An elastic analysis object is represented as an aggregate of a plurality of particles, and when solving the equation of motion for each particle to simulate the behavior of the analysis object, a solution can be obtained at high speed.

### General FIRE

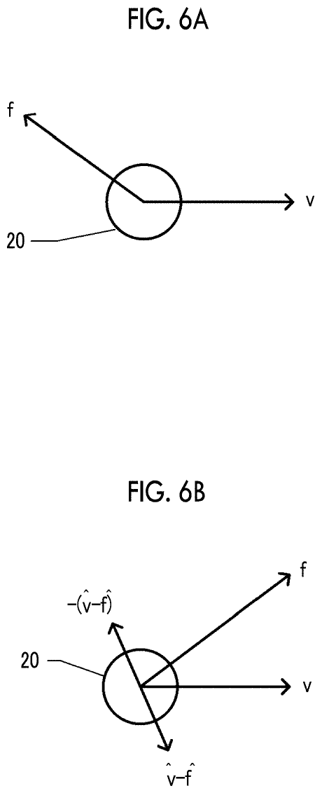

Before explaining the embodiment, FIRE, which is a method for obtaining a solution at high speed in a simulation using a molecular dynamics, will be described with reference to FIGS. 6A and 6B.

FIGS. 6A and 6B are schematic diagrams of an analysis model analyzed by applying FIRE. A particle 20 is disposed in the analysis space. The velocity of the particle 20 is marked as v, and the force acting on the particle 20 is marked as f. When applying FIRE, the following determination value P(t) is calculated for each time step.

P(t)=f(t)·v(t) (1)

When the determination value P(t) is negative or zero, that is, when the angle between the velocity v and the force f is 90° or more as shown in FIG. 6A, the velocity v(t+Δt) of the particle 20 in the next state is determined as follows.

v(t+Δt)=0, (P(t)≤0) (2)

In other words, when the force acts in the direction of decelerating the particle 20, the velocity of the particle 20 in the next state is set to zero.

When the determination value P(t) is positive, that is, when the angle between the velocity v and the force f is less than 90° as shown in FIG. 6B, the following equation of motion is solved for the particle 20 to obtain the velocity v(t+Δt) in the next state.

$$\dot{v} ( t ) = \frac{f ( t )}{m} - γ ( t ) \vert v ( t ) \vert \cdot \{ \hat{v} ( t ) - \hat{f} ( t ) \} , ( P ( t ) > 0 ) \qquad ( 3 )$$

Here, m is the mass of the particle 20, and γ is the attenuation constant. The hat attached to the vector means that it is a unit vector. Expression (3) means that the particle 20 is further accelerated in the direction of the force f.

### FIRE by Reference Example

Next, FIRE by a reference example in which an elastic analysis object is applied to an analysis model represented by an aggregate of particles will be described.

In the FIRE according to the reference example, the determination value P(t) is defined by the following expression.

$$P ( t ) = {\sum }_{i}^{N} {f}_{i} ( t ) \cdot {v}_{i} ( t ) \qquad ( 4 )$$

Here, fᵢ is the force acting on the i-th particle, and vᵢ is the velocity of the i-th particle. N is the number of particles, and Σ on the right side of Expression (4) means that all the particles constituting the analysis object are totaled.

When the determination value P(t) is negative or zero, the velocity vᵢ(t+Δt) of the i-th particle 20 in the next state is determined as follows.

vᵢ(t+Δt)=0, (P(t)≤0) (5)

When the determination value P(t) is positive, the velocity vᵢ(t+Δt) in the next state is obtained by solving the following equation of motion for the i-th particle 20.

$${\dot{v}}_{i} ( t ) = \frac{{f}_{i} ( t )}{{m}_{i}} - γ ( t ) \vert {v}_{i} ( t ) \vert \cdot \{ {\hat{v}}_{i} ( t ) - {\hat{f}}_{i} ( t ) \} , ( P ( t ) > 0 ) \qquad ( 6 )$$

Here, mᵢ is the mass of the i-th particle.

The present inventors have recognized that the FIRE according to the above reference example has the follows.

In the FIRE according to the reference example, as shown in Expression (4), the total value of the inner product of the force acting on each of the particles and the velocity of the particles is used as the determination value P(t). Therefore, even when the velocity of the center of gravity of the analysis object is zero, the determination value P(t) is not zero due to the internal oscillation, but has a certain value. When the temperature of the analysis object is high or the velocity of the analysis object approaches convergence, the condition that the determination value P(t) is negative is often satisfied. When this condition is satisfied, the velocity of each particle becomes zero, which causes a delay in convergence.

Further, when the determination value P(t) is positive, as shown in Expression (6), the equation of motion to which the force accelerating in the direction of the force is additionally applied to each particle is solved. The attenuation constant γ (t) needs to be set for each of the particles 20. Since the mass and the interaction with the surroundings are different between the particles 20, it is difficult to set the attenuation constant γ, and the calculation may fail.

### Embodiment

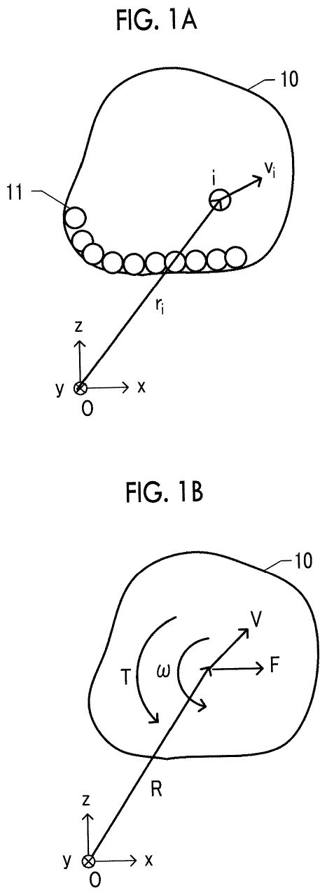

The simulation method, simulation apparatus, and program according to one embodiment of the present invention will be described with reference to FIGS. 1A to 5.

FIG. 1A is a schematic diagram showing an analysis model in which an elastic analysis object 10 is represented by an aggregate of a plurality of particles 11. Note that FIG. 1A shows only a part of the particles 11 constituting the analysis object 10. The xyz orthogonal coordinate system is defined for the analysis space, the position vector of the i-th particle 11 is marked as rᵢ, and the velocity of the i-th particle 11 is marked as vᵢ.

In the embodiment, the elastic analysis object 10 is assumed to be one rigid body, and the physical quantity is defined for the analysis object 10 regarded as a rigid body.

FIG. 1B is a schematic diagram showing a physical quantity defined for the analysis object 10 regarded as a rigid body. The position vector of the center of gravity of the analysis object 10 is marked as R. The translational force acting on the center of gravity of the analysis object 10 is marked as F, and the torque when the center of gravity is the center of rotation is marked as T. The translational velocity of the center of gravity of the analysis object 10 is marked as V, and the angular velocity is marked as ω. For example, when the analysis object such as a metal member is deformed only slightly, the analysis object is regarded as a rigid body, and the translational force F, torque T, translational velocity V, and angular velocity ω applied to the analysis object can be defined.

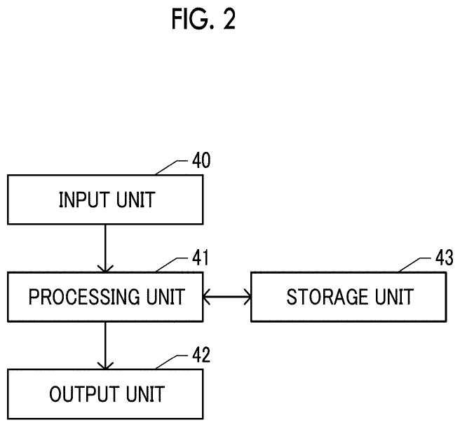

FIG. 2 is a block diagram of the simulation apparatus according to the present embodiment. The simulation apparatus according to the present embodiment includes an input unit 40, a processing unit 41, an output unit 42, and a storage unit 43. Simulation conditions and the like are input from the input unit 40 to the processing unit 41. Further, various commands are input from the user to the input unit 40. Examples of the input unit 40 include a communication device, a removable media reading device, a keyboard, a pointing device, and the like.

The processing unit 41 executes a simulation by the molecular dynamics, based on the input simulation conditions and commands. Further, the simulation result is output to the output unit 42. Examples of the simulation result include information representing temporal changes of the positions of the particles 11 (FIG. 1A) constituting the simulation object. Examples of the processing unit 41 include a central processing unit (CPU) of a computer. A program for causing the computer to execute the simulation by the molecular dynamics is stored in the storage unit 43. Examples of the output unit 42 include a communication device, a removable media writing device, a display, and the like.

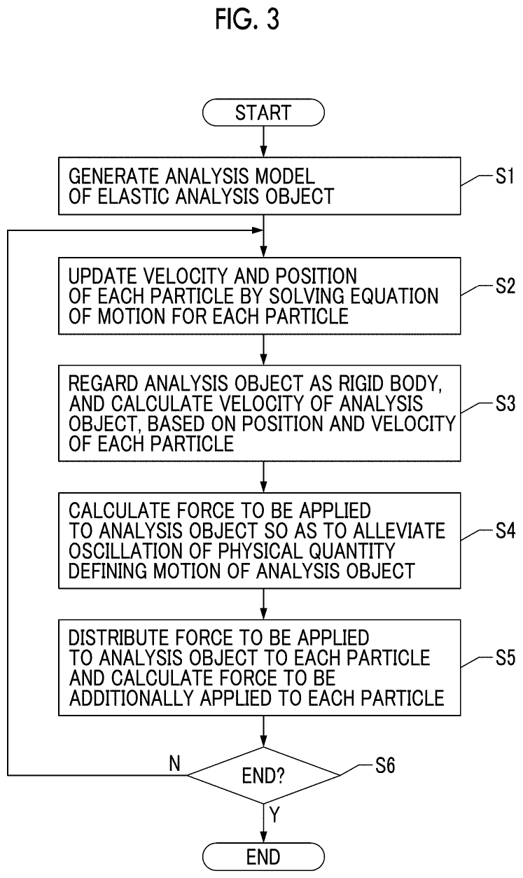

FIG. 3 is a flowchart of the simulation method according to the present embodiment. By executing the program stored in the storage unit 43 (FIG. 2) by the processing unit 41 (FIG. 2) , the process of each step of the simulation method is executed.

First, the processing unit 41 generates an analysis model in which the elastic analysis object 10 (FIG. 1A) is represented by an aggregate of a plurality of particles, based on the simulation conditions input from the input unit 40 (step S1). The simulation conditions include the shape of the analysis object 10, information that depends on the physical property value of the analysis object 10, the initial condition and boundary condition of simulation, a time step width, an analysis end condition, and the like. The information that depends on the physical property value of the analysis object 10 includes information that defines the interaction potential between particles in the analysis model.

Next, the equation of motion is solved for each particle 11 (FIG. 1A), and the velocity and position of each particle 11 are updated by one time step.

Next, the analysis object 10 (FIG. 1B) is regarded as a rigid body, and the force acting on the analysis object 10 and the velocity of the analysis object 10 are calculated based on the position and velocity of the particles 11 (step S3). The velocity of the analysis object 10 includes a translational velocity of the center of gravity and an angular velocity with the center of gravity as the center.

The translational force F acting on the analysis object 10 is calculated by the following expression.

$$F = {\sum }_{i}^{N} {f}_{i} \qquad ( 7 )$$

That is, the translational force F acting on the analysis object 10 is the vector sum of the forces fᵢ acting on each of the particles 11.

The translational velocity V of the analysis object 10 is calculated by the following expression.

$$V = \frac{1}{M} {\sum }_{i}^{N} {m}_{i} {v}_{i} \qquad ( 8 )$$

Here, M is the sum of the masses of all the particles 11 constituting the analysis object 10. The translational velocity V of the analysis object 10 is a value obtained by averaging the velocity vᵢ of the particles 11 constituting the analysis object 10 weighted by the mass mᵢ of the particles 11.

The torque T=(Tₓ, T_{y}, T_{z}) acting on the analysis object 10 is calculated by the following expression.

$${T}_{x} = {{\sum }_{i}}^{N} ( {y}_{i} {f}_{i , z} - {z}_{i} {f}_{i , y} ) \\ {T}_{y} = {\sum }_{i}^{N} ( {z}_{i} {f}_{i , x} - {x}_{i} {f}_{i , x} ) \\ {T}_{z} = {\sum }_{i}^{N} ( {x}_{i} {f}_{i , y} - {y}_{i} {f}_{i , x} ) \qquad ( 9 )$$

Here, Tₓ, T_{y}, and T_{z} are the x component, the y component, and the z component of the torque T acting on the analysis object 10, respectively. xᵢ, yᵢ, and zᵢ are the x-coordinate, the y-coordinate, and the z-coordinate of the i-th particle 11 when the center of gravity of the analysis object 10 is the origin, respectively. f_{i,x}, f_{i,y}, and f_{i,z} are the x component, the y component, and the z component of the force fᵢ acting on the i-th particle 11, respectively.

The angular velocity ω=(ωₓ, ω_{y}, and ω_{z}) of the analysis object 10 is calculated by the following expression.

$${ω}_{x} = \frac{1}{{I}_{x}} {\sum }_{i}^{N} {m}_{i} ( {y}_{i}^{2} + {z}_{i}^{2} ) ( {y}_{i} {v}_{i , z} - {z}_{i} {v}_{i , y} ) \\ {ω}_{y} = \frac{1}{{I}_{y}} {\sum }_{i}^{N} {m}_{i} ( {z}_{i}^{2} + {x}_{i}^{2} ) ( {z}_{i} {v}_{i , x} - {x}_{i} {v}_{i , z} ) \\ {ω}_{z} = \frac{1}{{I}_{z}} {\sum }_{i}^{N} {m}_{i} ( {x}_{i}^{2} + {y}_{i}^{2} ) ( {x}_{i} {v}_{i , y} - {y}_{i} {v}_{i , x} ) \qquad ( 10 )$$

Here, ωₓ, ω_{y}, and ω_{z} are the x component, the y component, and the z component of the angular velocity ω, respectively. v_{i,x}, v_{i,y}, and v_{i,z} are the x component, the y component, and the z component of the velocity of the i-th particle 11, respectively.

Iₓ, I_{y}, and I_{z} are moments of inertia around the x-axis, y-axis, and z-axis, respectively, and are defined by the following expression.

$${I}_{x} = {\sum }_{i}^{N} {m}_{i} ( {y}_{i}^{2} + {z}_{i}^{2} ) \\ {I}_{y} = {\sum }_{i}^{N} {m}_{i} ( {z}_{i}^{2} + {x}_{i}^{2} ) \\ {I}_{z} = {\sum }_{i}^{N} {m}_{i} ( {x}_{i}^{2} + {y}_{i}^{2} ) \qquad ( 11 )$$

After step S3 (FIG. 3), the processing unit 41 (FIG. 2) applies FIRE to the motion of the analysis object 10 so as to alleviate the oscillation of the physical quantity defining the motion of the analysis object 10 regarded as a rigid body. Thus, the force to be applied to the analysis object 10 regarded as a rigid body is calculated. More specifically, the translational force and the torque to be applied to the analysis object 10 are calculated (step S4).

Hereinafter, the process of step S4 will be described in detail.

The motion of the analysis object 10 is separated into translational motion and rotational motion, and FIRE is applied to each motion. First, the FIRE applied to the translational motion will be described.

The determination value Pₜ(t) of FIRE applied to the translational motion is defined as follows.

Pₜ(t)=F(t)·V(t) (12)

This expression is obtained by replacing the force f acting on the particles in the determination value (Expression (1)) of FIRE applied to the individual particles with the translational force F acting on the analysis object 10, and replacing the velocity v of the particles with the translational velocity V of the analysis object 10.

In a case where FIRE is applied to the translational motion of the analysis object 10, when the determination value Pₜ(t) is zero or negative, the translational velocity V(t+Δt) of the analysis object 10 in the next state may be determined as follows.

V(t+Δt)=0, (P(t)≤0) (13)

Expression (13) corresponds to Expression (2) in which the velocity v of the particles is set to zero.

When the determination value Pₜ(t) is positive, the translational velocity V(t+Δt) in the next state may be obtained by solving the following equation of motion for the analysis object 10.

$$\hat{V} ( t ) = \frac{F ( t )}{M} - α \vert V ( t ) \vert \cdot \{ \hat{V} ( t ) - \hat{F} ( t ) \} , ( {P}_{t} ( t ) > 0 ) \qquad ( 14 )$$

Expression (14) corresponds to the equation of motion for particles (Expression (3)). Here, α is an attenuation constant.

The determination value Pᵣ(t) of FIRE applied to the rotational motion is defined as follows.

Pᵣ(t)=T(t)·ω(t) (15)

Expression (15) is obtained by replacing the translational force F and the translational velocity V of Expression (12) with respect to the translational motion with the torque T and the angular velocity ω, respectively.

Ina case where FIRE is applied to the rotational motion of the analysis object 10, when the determination value Pᵣ(t) is zero or negative, the angular velocity ω(t+Δt) of the analysis object 10 in the next state may be determined as follows.

ω(t+Δt)=0, (Pᵣ(t)≤0) (16)

Expression (16) corresponds to Expression (13) in which the translational velocity V of the analysis object 10 is set to zero.

When the determination value Pᵣ(t) is positive, the angular velocity ω(t+Δt) in the next state may be obtained by solving the following equation of motion for the analysis object 10.

$$\dot{ω} ( t ) = \frac{T ( t )}{I} - β \vert ω ( t ) \vert \cdot \{ \hat{T} ( t ) - \hat{ω} ( t ) \} , ( {P}_{r} ( t ) > 0 ) \qquad ( 17 )$$

Expression (17) corresponds to the equation of motion for translational motion (Expression (14)). Here, β is an attenuation constant. I is the moment of inertia of the analysis object 10 and is defined by Expression (11). Here, T(t)/I means a vector (Tx/Ix, Ty/Iy, Tz/Iz).

From Expression (14), the translational force F_{add} to be additionally applied to the analysis object 10 by FIRE is described by the following expression.

F_{add}=−αM|V(t)|·{\hat{V}(t)−\hat{F}(t)} (18)

From Expression (17), the x component of the torque T_{add} to be additionally applied to the analysis object 10 by FIRE is described by the following expression. The same applies to the y component and the z component of the torque T_{add}.

T_{add,x}=−βIₓ|ω(t)|·[\hat{T}(t)−\hat{ω}(t)]ₓ (19)

After step S4, the processing unit 41 distributes the translational force F_{add} and the torque T_{add} to be additionally applied to the analysis object 10 to each particle 11, and the force to be additionally applied to the particles 11 is calculated (step S5).

The translational force F_{add} to be additionally applied is distributed to each particle 11 based on the following expression.

$${f}_{i} = \frac{F}{N} \qquad ( 20 )$$

The x component t_{i,x}, the y component t_{i,y}, and the z component t_{i,z} of the force to be applied to the i-th particle 11 when the torque T_{add} to be additionally applied is distributed to each particle 11 are described by the following expression.

$${t}_{i , x} = \frac{{m}_{i} ( {y}^{2} + {z}^{2} )}{{I}_{x}} {T}_{add , x} \\ {t}_{i , y} = \frac{{m}_{i} ( {z}^{2} + {x}^{2} )}{{I}_{x}} {T}_{add , y} \\ {t}_{i , x} = \frac{{m}_{i} ( {x}^{2} + {y}^{2} )}{{I}_{x}} {T}_{add , z} \qquad ( 21 )$$

When the determination value Pₜ(t) for the translational motion of the analysis object 10 is zero or negative, the translational velocity V defined by Expression (8) becomes zero, by subtracting the translational velocity V of the analysis object 10 at the present time from the velocity vᵢ of each of the particles 11. When the determination value Pᵣ(t) for the rotational motion is zero or negative, ωₓ, ω_{y}, and ω_{z} of Expression (10) become zero, by subtracting the velocity rᵢω of each of the particles 11 corresponding to the rotational velocity of the analysis object 10, from the velocity vᵢ of each of the particles 11.

The procedure from step S2 to step S5 is repeated until the analysis end condition is satisfied (step S6). When solving the equation of motion in step S2, the force to be additionally applied, obtained in step S5, is added to each of the particles 11.

Next, the excellent effects of the above embodiment will be described.

In the above embodiment, in step S4 (FIG. 3), since FIRE is applied to the motion of the analysis object 10 regarded as a rigid body, the calculation time until the analysis object 10 as a whole reaches an equilibrium state can be shortened.

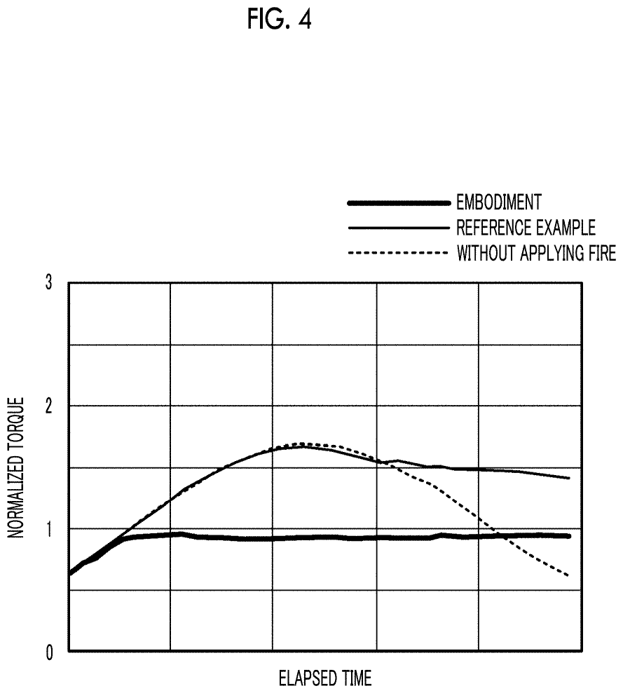

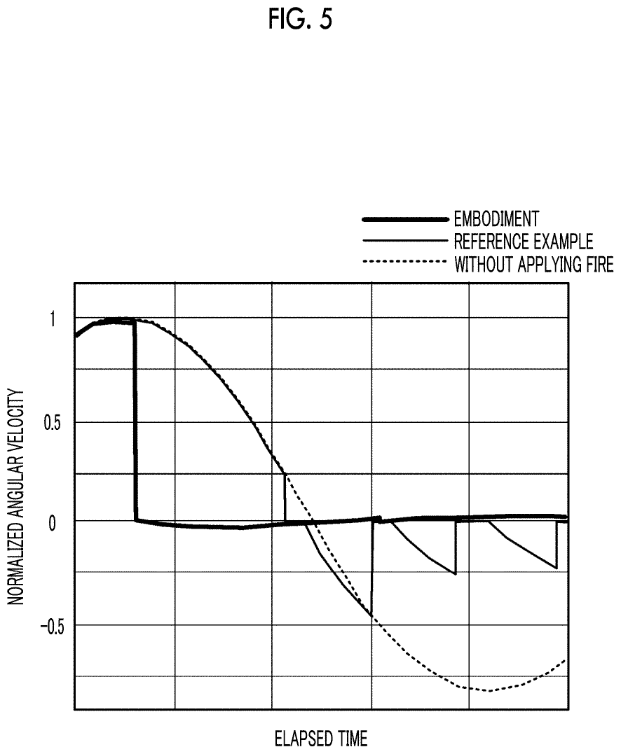

In order to confirm the excellent effect of the above embodiment, a static analysis of a cylindrical rotating body was performed using the simulation method according to the above embodiment. Next, the simulation results will be described with reference to FIGS. 4 and 5. In the simulation, a part of the region of one end surface of the rotating body is attached to the fixing member and fixed, and a constant torque is applied to the outer peripheral surface of the rotating body.

FIG. 4 is a graph showing a temporal change of a torque applied to a fixing member that fixes a part of a region of one end surface of a rotating body. The horizontal axis represents the elapsed time from the time when a certain time has elapsed from the start of analysis, and the vertical axis represents the torque normalized with the applied torque. The thick solid line in FIG. 4 indicates the torque obtained by using the simulation method according to the above embodiment, the thin solid line indicates the torque obtained by using the simulation method according to the reference example using Expressions (4) to (6), and the broken line indicates the torque obtained by simulation without applying FIRE. In the equilibrium state, the torque applied to the fixing member is equal to the applied torque. That is, the normalized torque becomes 1.

When FIRE is not applied, the calculated value of torque oscillates around an equilibrium value. The calculated value of torque according to the reference example gradually approaches the equilibrium value after overshooting. On the other hand, the calculated value of torque according to the above embodiment is a substantially equilibrium value, the calculated value of torque gradually increases, and the calculated value of torque reaches a substantially equilibrium state in a short time.

FIG. 5 is a graph showing a temporal change of an angular velocity co when the rotating body is regarded as a rigid body. The horizontal axis represents the elapsed time from the time when a certain time has elapsed from the start of analysis, and the vertical axis represents the angular velocity normalized with the maximum value set to 1. The thick solid line in FIG. 4 indicates the angular velocity obtained by using the simulation method according to the above embodiment, the thin solid line indicates the angular velocity obtained by using the simulation method according to the reference example using Expressions (4) to (6), and the broken line indicates the angular velocity obtained by simulation without applying FIRE. In the equilibrium state, the angular velocity becomes zero.

When FIRE is not applied, the calculated value of the angular velocity oscillates around zero. The calculated value of the angular velocity according to the reference example decreases after reaching the maximum, and becomes zero discontinuously immediately before the equilibrium state. When the calculated value of the angular velocity becomes zero discontinuously, the determination value P(t) of Expression (4) is zero or negative. On the other hand, in the calculated value of the angular velocity according to the above embodiment, the calculated value of the angular velocity becomes zero discontinuously immediately after the calculated value of the angular velocity becomes a substantially maximum value. When the calculated value of the angular velocity becomes zero discontinuously, the determination value Pₜ(t) of Expression (12) or the determination value Pᵣ(t) of Expression (15) is zero or negative. In the present simulation, the determination value Pᵣ(t) is zero or negative.

As shown in FIGS. 4 and 5, by using the simulation method according to the above embodiment, it can be seen that the time required to reach the equilibrium state is shortened, as compared to the case where FIRE is not applied and the case where the simulation method according to the reference example is used.

Next, a modification example of the above embodiment will be described.

In the above embodiment, in step S3 (FIG. 3), the translational velocity and the angular velocity are obtained as the velocity of the analysis object 10 regarded as a rigid body, but only one of the translational velocity and the angular velocity may be obtained, and FIRE may be applied to only one of the translational motion and the rotational motion of the analysis object 10.

The above-described embodiment is an example, and the present invention is not limited to the above-described embodiment. For example, it will be apparent to those skilled in the art that various modifications, improvements, combinations, and the like can be made.

It should be understood that the invention is not limited to the above-described embodiment, but may be modified into various forms on the basis of the spirit of the invention. Additionally, the modifications are included in the scope of the invention.

What is claimed is:

1. A simulation method that represents an elastic analysis object as an aggregate of a plurality of virtual particles,
obtains a force acting on each particle at each time step, and
updates a velocity and a position of each particle by solving an equation of motion for each particle, based on the force acting on each particle, the simulation method comprising:
when solving the equation of motion for each particle,
regarding the analysis object as a rigid body, and calculating a force acting on the analysis object and a velocity of the analysis object, based on the force acting on each particle obtained at each time step and the velocity and the position of each particle;
applying FIRE to a motion of the analysis object to calculate a force to be applied to the analysis object;
obtaining a force to be additionally applied to each particle, by distributing the force to be applied to the analysis object to the plurality of particles; and
solving the equation of motion, by adding the force to be additionally applied, to the force acting on each particle.

2. The simulation method according to claim 1, wherein
a translational force and a torque are calculated as the force acting on the analysis object,
a translational velocity and an angular velocity are calculated as the velocity of the analysis object, and
when applying the FIRE to the motion of the analysis object, the FIRE is applied to each of a translational motion and a rotational motion of the analysis object to calculate the translational force and the torque as the force to be applied to the analysis object.

3. A simulation apparatus that simulates a behavior of an elastic analysis object using a molecular dynamics method, the simulation apparatus comprising:
an input unit to which a simulation condition is input; and
a processing unit that performs analysis, based on the simulation condition input to the input unit, wherein
the processing unit
represents the analysis object as an aggregate of a plurality of virtual particles,
obtains a force acting on each particle at each time step,
updates a velocity and a position of each particle by solving an equation of motion for each particle, based on the force acting on each particle, and
when solving the equation of motion for each particle,
regards the analysis object as a rigid body, and calculates a force acting on the analysis object and a velocity of the analysis object, based on the force acting on each particle obtained at each time step and the velocity and the position of each particle;
applies FIRE to a motion of the analysis object to calculate a force to be applied to the analysis object,
obtains a force to be additionally applied to each particle, by distributing the force to be applied to the analysis object to the plurality of particles, and
solves the equation of motion, by adding the force to be additionally applied, to the force acting on each particle.

4. The simulation apparatus according to claim 3, wherein
the processing unit calculates a translational force and a torque, as the force acting on the analysis object,
calculates a translational velocity and an angular velocity, as the velocity of the analysis object, and
when applying the FIRE to the motion of the analysis object, applies the FIRE to each of a translational motion and a rotational motion of the analysis object to calculate the translational force and the torque as the force to be applied to the analysis object.

5. A non-transitory computer readable medium storing a program causing a computer to execute a process comprising:
acquiring a simulation condition;
representing an elastic analysis object as an aggregate of a plurality of virtual particles;
obtaining a force acting on each particle at each time step;
updating a velocity and a position of each particle by solving an equation of motion for each particle, based on the force acting on each particle; and
when solving the equation of motion for each particle,
regarding the analysis object as a rigid body, and calculating a force acting on the analysis object and a velocity of the analysis object, based on the force acting on each particle obtained at each time step and the velocity and the position of each particle;
applying FIRE to a motion of the analysis object to calculate a force to be applied to the analysis object;
obtaining a force to be additionally applied to each particle, by distributing the force to be applied to the analysis object to the plurality of particles; and
solving the equation of motion, by adding the force to be additionally applied, to the force acting on each particle.
